(19) United States
(12) Patent Application Publication
(10) Pub. No.: US 2015/0047532 A1
(43) Pub. Date: Feb. 19, 2015

(54) SYNTHETIC SPIDER SILK PROTEIN COMPOSITIONS AND METHODS

(71) Applicant: Utah State University, North Logan, UT (US)

(72) Inventors: Randolph V. Lewis, Nibley, UT (US); Justin A. Jones, Nibley, UT (US)

(21) Appl. No.: 14/459,244

(22) Filed: Aug. 13, 2014

Related U.S. Application Data
(60) Provisional application No. 61/865,487, filed on Aug. 13, 2013.

(51) Int. Cl. C07K 14/435; B29D 99/00; B29C 47/00; C09D 189/00; C08L 89/00

(52) U.S. Cl. CPC C07K 14/43563; C09D 189/00; C08L 89/00; B29C 47/0057; B29D 99/0078; B29K 2089/00; B29L 2031/731
USPC 106/124.4; 530/353; 516/103; 516/16; 264/164

(57) ABSTRACT

A method for solubilizing recombinant spider silk proteins in an aqueous solutions, where the method includes mixing recombinant spider silk proteins with water to form a mixture and heating the mixture in a closed vessel to form a solution.

### TECHNICAL FIELD

The present disclosure relates to synthetic spider silk protein compositions. More specifically, the present disclosure relates to aqueous solutions of recombinant spider silk proteins (“rSSP”) and synthetic spider silk protein compositions made from such aqueous solutions.

### SUMMARY

The present disclosure in aspects and embodiments addresses these various needs and problems by providing a method of solubilizing recombinant spider silk proteins (rSSP) in an aqueous solution and related compositions. Exemplary methods include mixing rSSP with water to form a mixture, and microwaving the resulting mixture to form a solution. Optional steps also include, sonicating the mixture, centrifuging the solution, sonicating the solution, and adding various additives to the mixture. Suitable additives may be configured to decrease gel formation of the solution.

In one aspect, a method of solubilizing one or more recombinant spider silk proteins in an aqueous solution is disclosed, which includes mixing the one or more recombinant spider silk proteins with water to form a mixture in a sealed container and heating the mixture to form a solution.

In some embodiments, heating is performed with microwave irradiation. In some embodiments, the method includes sonicating the mixture. In some embodiments, the method includes sonicating the solution. In some embodiments, the method includes centrifuging the solution.

In some embodiments, the method includes providing additives for reducing gel formation in the solution. In some embodiments, the additives are selected from the group consisting of: an acid, a base, free amino acids, surfactants, and combinations thereof. In some embodiments, the additives are selected from the group consisting of: propionic acid, formic acid, acetic acid, ammonium hydroxide, L-arginine, L-glutamic acid, β-mercaptoethanol, dithiothreitol, and combinations thereof.

In some embodiments, the one or more recombinant spider silk proteins are selected from the group consisting of: M4, M5, MaSP1, a MaSP1 analogue, MaSP2, an MaSP2 analogue, and combinations thereof.

In some embodiments, the ratio of the one or more recombinant spider silk proteins to water in the mixture is from 1:10 to 1:2.

In some embodiments, the method includes obtaining a recombinant spider silk protein fiber from the mixture. In some embodiments, the method includes stretching the fiber. In some embodiments, the method includes obtaining a recombinant spider silk protein material from the mixture. In some embodiments, the method includes stretching the material.

In another aspect, recombinant spider silk protein materials prepared using techniques disclosed herein, where the material has the form of a hydrogel, lyogel, film, coating, foam, fiber, and combinations thereof. In some embodiments, the material is a hydrogel. In some embodiments, the material is a lyogel. In some embodiments, the material is a film. In some embodiments, the material is a coating. In some embodiments, the material is a foam. In some embodiments, the material is a fiber.

In another aspect, an aqueous solution of recombinant spider silk proteins is disclosed having one or more recombinant spider silk proteins and water, wherein the amount of the one or more recombinant spider silk proteins is greater than about 2% w/v. In some embodiments, the amount of the one or more recombinant spider silk proteins is less than about 50% w/v.

In some embodiments, the solution includes one or more additives for reducing gel formation. In some embodiments, the one or more additives are selected from the group consisting of: an acid, a base, free amino acids, surfactants, and combinations thereof. In some embodiments, the one or more additives are selected from the group consisting of: propionic acid, formic acid, acetic acid, ammonium hydroxide, L-arginine, L-glutamic acid, β-mercaptoethanol, dithiothreitol, and combinations thereof.

### BRIEF DESCRIPTION OF THE FIGURES

FIG. 1A illustrates an exemplary recombinant spider silk fiber prepared according to one embodiment.

FIG. 1B illustrates an exemplary recombinant spider silk fiber prepared according to one embodiment.

FIG. 2 is a schematic representation of gluing boards used to characterize one embodiment.

FIG. 3 is the hysteresis testing results on hydrogels according to one embodiment.

FIG. 4 is a silicon wafer Purple (darkest shading) spider silk coated silicon wafer with methyl violet; next clockwise: control with spider silk coating and without kanamycin; next clockwise: Spider silk coating with 50 mg/L kanamycin; next clockwise: spider silk coating with 250 mg/L kanamycin; next clockwise: spider silk coating with 500 mg/L kanamycin. Bacterial lawn is E. coli XL-1 blue cells.

FIG. 5 is depiction showing silicon urinary catheters (3 french) coated with spider silk protein (top), spider silk coating loaded with 50 mg/L kanamycin (middle) and spider silk coating loaded with 500 mg/L kanamycin (bottom) according to one embodiment on a lawn of bacteria.

FIG. 6 is depiction showing a stainless steel plate that was dip coated with recombinant spider silk protein according to one embodiment.

### DETAILED DESCRIPTION

The present disclosure covers methods, compositions, reagents, and kits for making aqueous solutions of rSSP and for synthetic spider silk protein compositions derived from such solutions.

In the following description, numerous specific details are provided for a thorough understanding of specific preferred embodiments. However, those skilled in the art will recognize that embodiments can be practiced without one or more of the specific details, or with other methods, components, materials, etc. In some cases, well-known structures, materials, or operations are not shown or described in detail in order to avoid obscuring aspects of the preferred embodiments. Furthermore, the described features, structures, or characteristics may be combined in any suitable manner in a variety of alternative embodiments. Thus, the following more detailed description of the embodiments of the present invention, as illustrated in some aspects in the drawings, is not intended to limit the scope of the invention, but is merely representative of the various embodiments of the invention.

In this specification and the claims that follow, singular forms such as “a,” “an,” and “the” include plural forms unless the content clearly dictates otherwise. All ranges disclosed herein include, unless specifically indicated, all endpoints and intermediate values. In addition, “optional” or “optionally” refer, for example, to instances in which subsequently described circumstance may or may not occur, and include instances in which the circumstance occurs and instances in which the circumstance does not occur. The terms “one or more” and “at least one” refer, for example, to instances in which one of the subsequently described circumstances occurs, and to instances in which more than one of the subsequently described circumstances occurs.

The present disclosure covers methods, compositions, reagents, and kits for making aqueous solutions of rSSP and for synthetic spider silk protein compositions derived from such solutions.

rSSP's are conventionally dissolved in a very harsh organic solvent, 1,1,1,3,3,3-hexafluoroisopropanol (HFIP), to create “dopes” that can be used to create fibers, films, gels and foams. HFIP has been widely used and accepted as it is the only solvent that: 1) dissolves rSSP's at high concentrations (30% w/v) providing uniformity between various groups testing data, 2) is sufficiently volatile and miscible to be removed rapidly from the forming fiber, 3) leaves little to no residue behind that could interfere with fiber formation. In addition, rSSP's generally are insoluble in aqueous solutions after purification, necessitating an organic solvent that meets the criteria outlined in 1-3. However, there are significant problems with solvating rSSP's in HFIP or other organic solvents.

Dissolving rSSP in HFIP and then using pressure to extrude the dope into a coagulation bath does not allow the appropriate structures to form (notably β-sheets) to an extent that the fibers or films have to be post-spin processed multiple times to achieve protein structures that result in appreciable mechanical properties. See Lazaris et al., Spider Silk Fibers Spun from Soluble Recombinant Silk Produced in Mammalian Cells, Science 295, 472-476 (2002) (hereinafter “Lazaris”); and Teule et al., Modifications of spider silk sequences in an attempt to control the mechanical properties of the synthetic fibers, J. Mater Sci, 42, 8974-8985 (2007) (hereinafter “Teule”).

Such fiber processing methodologies include extruding the fiber into a coagulation bath that may include pure isopropanol or a mixture of isopropanol:water. The fiber may then be stretched (1.5-6×) in a second bath generally containing a mixture of isopropanol:water. A third bath may also be employed that contains pure water or a majority of water, and a second stretch applied in that bath. Water is the recurrent theme in these baths and it is the water that converts the helical structures present due to HFIP into strength providing β-sheets.

The cost of purchase and subsequent disposal of HFIP may be restrictive or prohibitive in an industrial setting of mass production. HFIP's cost of purchase is roughly $1,000/100 ml's of HFIP and 100 ml's of HFIP would likely be capable of solvating 20-30 g's of rSSP (20-30% w/v). Water is cheap even in its purest form. Per the MSDS published on Sigma Aldrich's web-site, disposal of HFIP requires; “Dissolve or mix the material with a combustible solvent and burn in a chemical incinerator equipped with an afterburner and scrubber,” a process that inherently has costs associated with it. Excess water can be evaporated or recycled and reused. Worker safety when utilizing such harsh, volatile solvents is also a consideration. Per the MSDS; “Material is extremely destructive to tissue of the mucous membranes and upper respiratory tract, eyes, and skin. Cough, Shortness of breath, Headache, Nausea” (SIC). Water has no such requirements. Finally, the process of producing rSSP products could not be considered “green” using HFIP.

rSSP's are largely insoluble in water. There are a few notable exceptions: Teule describes a series of proteins (Y₁S₈ and A₂S₈) that were produced in bacteria and purified via Ni⁺⁺ chromatography. Short fibers were pulled straight from the eluted, pure rSSP fraction. See Teule. Lazaris describes ADF-3 (Araneus diadematus MaSp1) produced in mammalian cell culture. Water soluble ADF-3 was concentrated in the presence of glycine and extruded into a coagulation bath. A final example is a series of recombinant aciniform-like synthetic proteins that were able to be spun from an aqueous solution very similar to Teule 2007 (Xu 2012). See Xu et al., Recombinant Minimalist Spider Wrapping Silk Proteins Capable of Native-Like Fiber Formation. PloS-One 7(11): e50227. Doi: 10.1371/journal.pone.0050227 (2012). However, outside of this small sub-set of rSSP's, water solubility is elusive. The majority of these proteins were much smaller than the natural proteins and thus are unlikely to make mechanically useful fibers.

U.S. Patent Application Publication No. 2011/0230911 filed by Amsilk utilizes a top down approach: genetic manipulations and expression system manipulations to try and create water soluble silk proteins. However, such processes are costly both in time to create the manipulations/cell lines and also in that the proteins appear to be expressed in mammalian cell cultures. The culture conditions for such cell lines are not only personnel and time intensive but also the ingredients and equipment are substantially more expensive than the more traditional bacterial expression systems. In addition, such methods are limiting as there are not that many iterations of various spider silk repeats that can be expressed in this manner that will result in a water soluble protein that has appreciable mechanical properties.

To address these and other challenges, this disclosure sets forth new and novel methods for solubilizing rSSP's in aqueous solutions and then creating resulting spider silk compositions therefrom. The methods and compositions described herein in embodiments create aqueous dopes from rSSPs that are otherwise not soluble in water. The methods and compositions described herein may be applied to proteins expressed by any organism, reducing the cost of production and also possibly improving the mechanical properties of the fibers, films, gels and foams by the inclusion of water in the dope.

In embodiments, methods of preparing aqueous dopes of rSSP may include the following steps: mixing rSSP, water, and optional additives; optionally sonicating the mixture; microwaving the mixture; and optionally centrifuging the microwaved mixture.

Aqueous Dopes

rSSP and water are combined to create a doping mixture of greater than about 2% w/v (e.g. 0.02 g SSpS:1 mL H₂O). In embodiments, the w/v does not typically exceed 50%. However, any percentage of less than 50% may be used.

Suitable rSSP's include: MaSp1 (as described in U.S. Pat. Nos. 7,521,228 and 5,989,894), MaSp2 (as described in U.S. Pat. Nos. 7,521,228 and 5,989,894), MiSp1 (as described in U.S. Pat. Nos. 5,733,771 and 5,756,677), MiSp2 (as described in U.S. Pat. Nos. 5,733,771 and 5,756,677), Flagelliform (as described in U.S. Pat. No. 5,994,099), chimeric rSSP's (as described in U.S. Pat. No. 7,723,109), Pyriform, aciniform, tubuliform, aggregate gland silk proteins, and AdF-3 and AdF-4 from araneus diadematus. Each of the above referenced patents is herein incorporated by reference in its entirety.

Dope Additives

Various optional additives may be optionally added to the mixture. Suitable additives include compositions that contribute to the solubility of the rSSP in the solution. Some additives break or weaken disulfide bonds, thereby increasing the solubility of rSSP's. Other additives also serve to prevent hydrogel formation after the completion of the microwave step, as set forth below. If the solution forms a hydrogel quickly and the desired end product is not a gel, then additives capable of delaying or inhibiting such a formation may be desirable. In some embodiments, multiple additives may be added to achieve desired end products.

For example, to combat hydrogel formation, various additives may be added to the suspension of rSSP and water prior to microwaving the suspension. In some embodiments, acid, base, free amino acids, surfactants, or combinations thereof may be employed to combat hydrogel formation. For example, additions of acid (formic acid and acetic acid alone or together at 0.1% to 10% v/v), base (ammonium hydroxide at 0.1% to 10% v/v), free amino acids (L-Arginine and L-Glutamic Acid at 1 to 100 mM) as well as a variety of surfactants (Triton X-100 at 0.1% v/v) may be used. The additions of these various chemicals not only aid the solubility of rSSP when microwaved but in certain combinations also delay the solution from turning into a hydrogel long enough for the solution to be spun into a fiber.

By altering and adjusting the combinations of additives to the dopes, the mechanical properties of the spun fiber are significantly impacted. For example, too much acid or base may result in fibers that are brittle with little to no extensibility; too little acid or base may result in dopes where the rSSP will not solubilize to the extent necessary for fiber spinning or turns to a hydrogel quickly.

Exemplary additives also include compositions capable of breaking or weakening disulfide bonds, such as β-mercaptoethanol or dithiothreitol may be added to reduce bonds and increase solubility. Suitable amounts of such additives may include from about 0.1 to about 5% (v/v). In embodiments where the rSSP does not contain cysteine, the use of such additives may be unnecessary. In some embodiments employing major ampulate silk proteins 1 and 2 (MaSp1 and MaSp2, respectfully), disulfide bonds (cysteine) are present in the C-terminus of the non-repetitive regions of MaSp1 and MaSp2. These proteins are described in U.S. Pat. Nos. 7,521,228 and 5,989,894, the entirety of which is herein incorporated by reference. In addition, the C-term is present in various goat-derived spider silk proteins M4, M5 and M55 proteins, which are described in U.S. Patent Application Publication No. 20010042255 A1, the entirety of which is incorporated by reference in its entirety. In some embodiments, formic acid and/or acetic acid may be included in as little as 0.3% (v/v) but even lower amounts (0.1% v/v) are possible. Additionally, it is possible to solubilize rSSP without using any additives.

Exemplary additives are set forth in Table 1 (below), where dope formulations prepared according to the methods described herein and their resultant fibers/films mechanical properties are listed in examples that follow.

TABLE 1
Additives

| | | 3 | 4 | |
|---|---|---|---|---|
| 1 | 2 | Free Amino | Disulfide | 5 |
| Acid | Base | Acids | Reduction | Other |
| Acetic | Ammonium | Arginine | β-mercapto- | Triton X-100 |
| | Hydroxide | | ethanol | |
| Formic | Sodium | Glutamic | Dithiothre- | Glutaral- |
| | Hydroxide | Acid | itol | dahyde |
| Trifluoroacetic | | Histidine | | Calcium |
| acid | | | | |
| Other Organic | | Glycine | | Potassium |
| Acids | | | | |
| Propionic | | Imidazole | | Other |
| Acid | | | | Surfactants |
| | | Other Free | | Other Ions |
| | | Amino Acids | | |

L-DOPA

In some embodiments, aqueous spin dopes omit additives. In some embodiments, the aqueous spin dope includes imidazole. In some embodiments, the aqueous spin dope includes propionic acid.

To formulate an aqueous solution of rSSP, additives can be chosen from any of the 5 columns. For instance one or a combination of acids can be chosen from column 1 and combined with one or combinations of free amino acids from column 3, as well as disulfide reducing compounds from column 4 and “Other” additives as required by the particular protein. Generally, it would not be useful to include both an acid from column 1 with a base from column 2 However, a base from column 2 can be combined with additives from columns 3-4.

Sonication

In some embodiments, the mixture containing water, rSSP's, and optional additives may be sonicated. The addition of sonication to the rSSP and water suspension may greatly increase the amount of solubilized protein. Sonication may be performed with any suitable sonicator, such as a Misonix 3000 with microtip at 3.0 watts) either prior to microwaving, after microwaving and cooling, or both. Thus in some embodiments, a solution formed containing water, rSSP's, and optional additives may be sonicated.

In embodiments, sonication may be employed to improve the amount of rSSP solubilized and, thus, reduce the amount of protein required to form an aqueous spin dope. Sonication also has the added benefit of producing a more homogenous solution. Sonication also improves and/or changes mechanical properties for rSSP composition products, particularly fiber mechanical properties.

For example, initial experiments required a 12.5% w/v MaSp1 analogue (125 mg MaSp1 into 1 ml of aqueous) in order to spin a fiber. Sonicating after microwaving reduced the concentration of MaSP1 to 5% w/v necessary to form fibers. Lower rSSP concentrations results in more fiber spun from a given amount of protein as well as finer fibers which has been demonstrated to increase the mechanical properties in other systems (electrospinning from HFIP based dope solutions).

Microwave Irradiation

The mixture containing water, rSSP's, and optional additives may be microwaved (or otherwise irradiated with microwave radiation) prior to or after the optional sonication step. In embodiments, any microwave may be employed. In some embodiments, the mixture should be sealed prior to microwaving so as to avoid evaporation.

The mixture may be microwaved (irradiated with microwaves) for any suitable amount of time to achieve the desired end product. The time depends on the power of the microwave and the amount of solution to be microwaved. In some embodiments, the solution may be stirred or agitated during microwaving so as to evenly expose the mixture to the microwaves. Appropriate times per unit being microwaved include, for example, from 10 to 90 seconds per 1 milliliter of mixture. In some embodiments the 1 ml mixture may be set at from about 10-100% power for from about 5 to 120 seconds.

Without wishing to be bound to any particularly mechanism or theory, it is believe that the irradiation of water and one or more rSSP's increases the temperature of the solution while concomitantly increasing the pressure on the constituents in solution when irradiated or otherwise heated in a sealed vessel.

After microwaving, the solution is allowed to cool and/or is taken to other processing steps, depending on the desired product.

Centrifugation

In some embodiments, the microwaved (irradiated) mixture may be optionally centrifuged. After centrifugation, the resulting supernatant may be removed and then used for rSSP compositions and further processing.

Gel Formation

Hydrogels may be generated from aqueous rSSP solutions by allowing the solubilized rSSP to cool. Additives to the dope such as acetic or formic acid can delay the formation of the hydrogel to allow the rSSP to be transferred to a mold prior to gelation. Theoretically, the variety of shapes that can be generated is limitless. The additives to the solution will change the mechanical properties of the resulting hydrogel. Hydrogel formation has been observed in solutions with as little as 3% w/v rSSP:water and all iterations greater than that. The higher the % of rSSP, the more rapidly the solution gelates. Work in other systems, Bombyx mori silk, has proven the phenomenon that increasing the ratio of silk to water improves the mechanical characteristics of the resulting hydrogel. As well, altering the temperature, pH and including calcium ions changes the properties of the gels (Kim, U J et al., 2004, Biomacromolecules “Structure and Properties of Silk Hydrogels” Biomacromolecules 5, 786-792).

An example of a hydrogel application is illustrated in Chao et al., “Silk Hydrogel for Cartilage Tissue Engineering.” Journal of Biomedical Materials Research Part B: Applied Biomaterials, Vol 95B, Issue 1 pg 84-90, 2010.

Aerogels may be formed by freezing and then lypholizing a solution or hydrogel of rSSP. Theoretically, the shapes for these aerogels is also limitless as their starting hydrogels could be allowed to form in a mold and then frozen and lyophilized.

Film Formation

Films may be produced by pouring a dope solution onto a substrate and allowing the water and other additives to evaporate. If it is desirable to remove the film from the substrate, PDMS or Teflon allow the removal of the films. A representative dope solution is: 50 mg/ml MaSP1 analogue, 1% Formic Acid, 1% acetic acid.

Films prepared by the techniques disclosed herein can vary in their dimensions. An exemplary film size in working embodiments covers a 30×7 mm are when the rSSP dope is poured. The film was then cut in half and the thick edges cut for a film with an average length and width of 15×5.5 mm and an average thickness of 25 um.

Resulting films can also be stretched in 50/50 isopropyl/water bath up to 3.5×. Resulting films can also be stretched in 80/20 MeOH/water bath up to 3×.

Films may be applied as coatings or utilized after removal from a substrate.

Foam Formation

Foam may be generated from aqueous based solvents by a variety of methods and dope conditions. One method reduced to practice is to formulate a dope solution similar/identical to that described for film generation. That solution is then placed into a vacuum chamber and a vacuum applied. The solution quickly expands and forms a foam upon curing in the chamber. Additives to the dopes such as surfactants will influence final cell size and further treatment of the foam (alcohol) are possible to also change the final properties of the foams. It is also possible that foams can be generated by chemical means, mainly peroxidase reactions, to produce CO₂ that creates bubbles in the dope and upon curing a foam remains. (See US 20110230911 A1 Scheibel). Again, this method is also influenced by additives such as surfactants and post formation treatments (alcohol). A final method is an extrusion method whereby the dope solution is mechanically mixed with air, or other gas, to produce foam. This method is also subject to additives and post formation treatments to alter the final foam product.

Fiber Formation

Fibers can be spun from aqueous solutions of rSSP by extrusion into a coagulation bath (alcohol) in a similar fashion as HFIP/aqueous based solutions of rSSP as described in US Patent Application Publication No. 2005/0054830. To summarize, the solubilized rSSP can be loaded into a syringe or other suitable extrusion instrument and then pushed through a fine bore needle into a bath comprised of isopropanol or other alcohol. As the rSSP drops through the alcohol, water is removed and a fiber is formed. That fiber can then be taken up or processed further by stretching in a second or even third bath comprised of alcohol(s), alcohol(s) and water or just water. Fibers have been formed from solutions with as little as 5% w/v solutions of rSSP:water. Similar 5% w/v solutions using HFIP as the solvent will not form fibers.

In some embodiments, it is unnecessary for the solution to remain liquid to form fibers. Indeed, in some embodiments, fibers may be formed from a hydrogel. For example, when forming fibers from MaSp2 proteins, the process may be stopped, the syringe immediately removed for visualization, and a hydrogel may be observed. In contrast, forming fibers from a hydrogel with MaSp1 proteins results in deleterious effects.

It is important to note that each individual rSSP, due to its unique amino acid sequence, will have different requirements for aqueous solubility. The rSSP concentration, microwave time and power setting, amount of acid or base, and requirements for free amino acids or surfactants will be different. There does not appear to be one set of additives that achieves aqueous solubility and that also delays hydrogel formation for all rSSP's.

As an example, a 12.5% w/v solution of a MaSp1 and MaSp2 analogue can be prepared identically in terms of additives. The MaSp1 will become soluble in water easily and stay liquid for an extended period of time. The MaSp2, on the other hand, will form a hydrogel within minutes of removal from the microwave and requires more microwave time to solubilize.

The following examples are illustrative only and are not intended to limit the disclosure in any way.

### EXAMPLES

### Process Example—Dope Preparation

An aqueous recombinant spider silk protein (rSSP) dope solution was prepared by weighing out the rSSP such that a 1-40% (w/v) of protein was achieved in 1 ml of water. For example, 50 mg's of protein in 1 ml of water yielded a 5% w/v solution of protein to water. The suspension of rSSP and water was sealed inside a 3 ml glass Wheaton vial using a PTFE lined cap. The suspension and vial were then placed in a conventional 1500 watt microwave and microwaved at 50% power for 30 seconds. This solubilized the protein powder in the water.

Although this method may work to solubilize the rSSP, the solution quickly formed a hydrogel upon cooling and was generally not available thereafter to spin fibers by extrusion. If the goal of generating the aqueous dope is to form films, foams, hydrogels or aerogels, this method may be acceptable. Microwave time may vary depending on the volume of the dope, rSSP used, additives chosen, and whether sonication is utilized.

### Process Example—Sonication

The following samples were prepared, one of which was not sonicated:

(1) Dope Not Sonicated (12.5% M4, 1% acetic acid, 1% Formic Acid, 50 mM L-Arg, Microwaved 30″ at 50% power, centrifuged at 6000 rpm for 3 minutes, 1.5× stretch, 40× objective);
(2) Dope Sonicated (5% M4, 1% acetic acid, 1% formic acid, 50 mM L-Arg., microwaved 35 sec. at 50% power, sonicated at power level 1.5 (3 Watts) for 1.5 min., microwaved 30 sec. at 50% power, centrifuged 1 min. at 6000 rpm, 1.5× stretch, 40× objective.

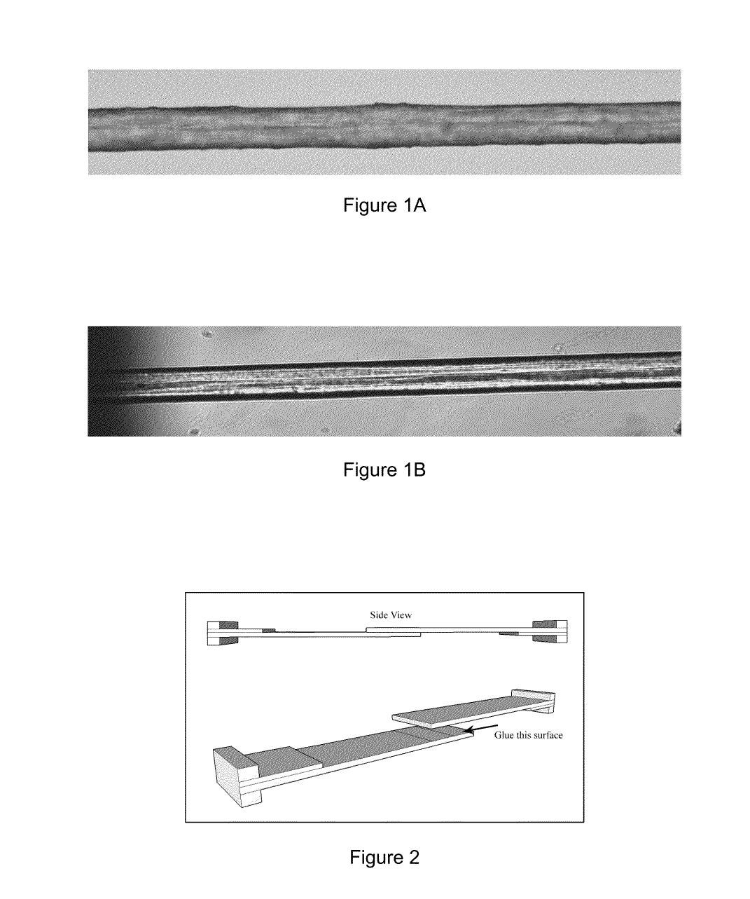

Fibers spun from dopes that are not sonicated (FIG. 1A), when analyzed microscopically, appear to have numerous lumps and discontinuities. The sonicated 5% w/v MaSP1 fibers (FIG. 1B) appear much more uniform. Sonication has the added benefit of requiring lower rSSP concentrations (5% compared to >8% without sonication) to spin fibers from. Lower concentrations are advantageous as less protein is used to spin similar lengths of fiber. Thus, fiber defects when spun from aqueous dopes may be diminished by sonication of the dope.

The following examples set forth numerous rSSP sample tests and resulting data according the formulations and processing criteria set forth below:

### Example Set 1

125 mg's of M5 (Nephila clavipes MaSP2 analogue) was measured out using a fine balance into a 3 ml Wheaton glass vial with PTFE seal inside a plastic cap. Included in the dope solution was 50 uL of 1M L-arginine (L-Arginine is prepared in 18.2 MOhm water), 50 uL of glacial acetic acid (5% v/v), and 900 uL of 18.2 MOhm water. The PTFE sealed cap was placed on the 3 ml vial tightly. The solution and vial were placed into a conventional microwave (GE 1.6 kW) and microwaved for 30 seconds at 50% power. After microwaving, the solution was placed into a centrifuge (VWR Clinical 2000 set at 6,000 RPM) for 2 minutes to clarify. The supernatant is removed from any remaining pellet for spinning fibers or producing other materials such as films, gels or foams.

Fiber testing results (10 samples) for 1.5× post spin stretch in an 80:20 isopropanol:water bath.

| | Diameter | Energy to Break | Max Stress | Max Strain |
|---|---|---|---|---|
| | (μm) | (MJ/m³) | (MPa) | (mm/mm) |
| Average | 37.25 | 0.75 | 60.05 | 0.02 |
| St. Dev. | 2.95 | 0.25 | 9.11 | 0.003 |

Fiber testing results (9 samples) for 2.0× post spin stretch in an 80:20 isopropanol:water bath.

| | Diameter | Energy to Break | Max Stress | Max Strain |
|---|---|---|---|---|
| | (μm) | (MJ/m³) | (MPa) | (mm/mm) |
| Average | 37.40 | 1.98 | 52.94 | 0.047 |
| St. Dev. | 2.27 | 1.43 | | 0.03 |

Fiber testing results (9 samples) for 2.5× post spin stretch in an 80:20 isopropanol:water bath.

| | Diameter | Energy to Break | Max Stress | Max Strain |
|---|---|---|---|---|
| | (μm) | (MJ/m³) | (MPa) | (mm/mm) |
| Average | 38.91 | 18.07 | 41.64 | 0.68 |
| St. Dev. | 5.15 | 14.64 | 17.17 | 0.54 |

Fiber testing results (10 samples) for 3.0× post spin stretch in an 80:20 isopropanol:water bath.

| | Diameter | Energy to Break | Max Stress | Max Strain |
|---|---|---|---|---|
| | (μm) | (MJ/m³) | (MPa) | (mm/mm) |
| Average | 26.71 | 40.25 | 84.54 | 0.57 |
| St. Dev. | 2.12 | 14.27 | 18.04 | 0.18 |

Fiber testing results (10 samples) for 3.5× post spin stretch in an 80:20 isopropanol:water bath.

| | Diameter | Energy to Break | Max Stress | Max Strain |
|---|---|---|---|---|
| | (μm) | (MJ/m³) | (MPa) | (mm/mm) |
| Average | 23.07 | 22.73 | 106.65 | 0.25 |
| St. Dev. | 2.64 | 8.76 | 22.91 | 0.09 |

### Example Set 2

125 mg's of M5 (Nephila clavipes MaSP2 analogue) was measured out using a fine balance into a 3 ml Wheaton glass vial with PTFE seal inside a plastic cap. Included in the dope solution was 50 uL of 1M L-Arginine (L-Arginine is prepared in 18.2 MOhm water), 100 uL of glacial acetic acid (10% v/v), and 850 uL of 18.2 MOhm water. The PTFE sealed cap was placed on the 3 ml vial tightly. The solution and vial were placed into a conventional microwave and microwaved for 30 seconds at 50% power. After microwaving, the solution was placed into a centrifuge (VWR Clinical 2000 set at 6,000 RPM) for 2 minutes to clarify. The supernatant was removed from any remaining pellet for spinning fibers or producing other materials such as films, gels or foams.

Fiber testing results (9 samples) 1.5× post spin stretch in an 80:20 isopropanol:water bath.

| | Diameter | Energy to Break | Max Stress | Max Strain |
|---|---|---|---|---|
| | (μm) | (MJ/m³) | (MPa) | (mm/mm) |
| Average | 34.17 | 4.52 | 72.88 | 0.07 |
| St. Dev. | 5.74 | 3.10 | 13.83 | 0.05 |

Fiber testing results (9 samples) 2.0× post spin stretch in an 80:20 isopropanol:water bath.

| | Diameter | Energy to Break | Max Stress | Max Strain |
|---|---|---|---|---|
| | (μm) | (MJ/m³) | (MPa) | (mm/mm) |
| Average | 31.02 | 3.92 | 74.05 | 0.08 |
| St. Dev. | 5.03 | 2.56 | 20.69 | 0.06 |

Fiber testing results (10 samples) 2.5× post spin stretch in an 80:20 isopropanol:water bath.

| | Diameter | Energy to Break | Max Stress | Max Strain |
|---|---|---|---|---|
| | (μm) | (MJ/m³) | (MPa) | (mm/mm) |
| Average | 25.95 | 15.34 | 102.62 | 0.19 |
| St. Dev. | 1.08 | 13.71 | 17.87 | 0.18 |

Fiber testing results (10 samples) 3.0× post spin stretch in an 80:20 isopropanol:water bath.

| | Diameter | Energy to Break | Max Stress | Max Strain |
|---|---|---|---|---|
| | (μm) | (MJ/m³) | (MPa) | (mm/mm) |
| Average | 25.71 | 41.89 | 87.67 | 0.55 |
| St. Dev. | 2.46 | 26.92 | 18.06 | 0.30 |

### Example Set 3

125 mg's of M5 (Nephila clavipes MaSP2 analogue) was measured out using a fine balance into a 3 ml Wheaton glass vial with PTFE seal inside a plastic cap. Included in the dope solution was 50 uL of 1M L-Arginine (L-Arginine is prepared in 18.2 MOhm water), 150 uL of glacial acetic acid (15% v/v), and 800 uL of 18.2 MOhm water. The PTFE sealed cap was placed on the 3 ml vial tightly. The solution and vial were placed into a conventional microwave and microwaved for 30 seconds at 50% power. After microwaving, the solution was placed into a centrifuge (VWR Clinical 2000 set at 6,000 RPM) for 2 minutes to clarify. The supernatant was removed from any remaining pellet for spinning fibers or producing other materials such as films, gels or foams.

Fiber testing results (10 samples) 1.5× post spin stretch in an 80:20 isopropanol:water bath.

| | Diameter | Energy to Break | Max Stress | Max Strain |
|---|---|---|---|---|
| | (μm) | (MJ/m³) | (MPa) | (mm/mm) |
| Average | 39.20 | 3.77 | 69.92 | 0.07 |
| St. Dev. | 10.74 | 3.66 | 15.36 | 0.06 |

Fiber testing results (10 samples) 2.0× post spin stretch in an 80:20 isopropanol:water bath.

| | Diameter | Energy to Break | Max Stress | Max Strain |
|---|---|---|---|---|
| | (μm) | (MJ/m³) | (MPa) | (mm/mm) |
| Average | 46.93 | 20.47 | 53.81 | 0.37 |
| St. Dev. | 5.23 | 23.18 | 14.17 | 0.35 |

Fiber testing results (10 samples) 2.5× post spin stretch in an 80:20 isopropanol:water bath.

| | Diameter | Energy to Break | Max Stress | Max Strain |
|---|---|---|---|---|
| | (μm) | (MJ/m³) | (MPa) | (mm/mm) |
| Average | 46.05 | 24.95 | 52.00 | 0.49 |
| St. Dev. | 6.42 | 25.25 | 16.49 | 0.43 |

### Example Set 4

125 mg's of M5 (Nephila clavipes MaSP2 analogue) was measured out using a fine balance into a 3 ml Wheaton glass vial with PTFE seal inside a plastic cap. Included in the dope solution was 50 uL of 1M L-Arginine (L-Arginine is prepared in 18.2 MOhm water), 200 uL of glacial acetic acid (20% v/v), and 750 uL of 18.2 MOhm water. The PTFE sealed cap was placed on the 3 ml vial tightly. The solution and vial were placed into a conventional microwave and microwaved for 30 seconds at 50% power. After microwaving, the solution was placed into a centrifuge (VWR Clinical 2000 set at 6,000 RPM) for 2 minutes to clarify. The supernatant was removed from any remaining pellet for spinning fibers or producing other materials such as films, gels or foams.

Fiber testing results (10 samples) 1.5× post spin stretch in an 80:20 isopropanol:water bath.

| | Diameter | Energy to Break | Max Stress | Max Strain |
|---|---|---|---|---|
| | (μm) | (MJ/m³) | (MPa) | (mm/mm) |
| Average | 46.38 | 0.26 | 33.18 | 0.014 |
| St. Dev. | 10.11 | 0.11 | 7.64 | 0.003 |

Fiber testing results (9 samples) 2.0× post spin stretch in an 80:20 isopropanol:water bath.

| | Diameter | Energy to Break | Max Stress | Max Strain |
|---|---|---|---|---|
| | (μm) | (MJ/m³) | (MPa) | (mm/mm) |
| Average | 44.65 | 1.09 | 63.71 | 0.02 |
| St. Dev. | 8.29 | 1.39 | 32.07 | 0.009 |

Fiber testing results (10 samples) 3.5× post spin stretch in an 80:20 isopropanol:water bath.

| | Diameter | Energy to Break | Max Stress | Max Strain |
|---|---|---|---|---|
| | (μm) | (MJ/m³) | (MPa) | (mm/mm) |
| Average | 37.44 | 8.44 | 80.85 | 0.13 |
| St. Dev. | 2.04 | 11.70 | 8.09 | 0.16 |

### Example Set 5

125 mg's of M4 (Nephila clavipes MaSP1 analogue) was measured out using a fine balance into a 3 ml Wheaton glass vial with PTFE seal inside a plastic cap. Included in the dope solution was 50 uL of 1M L-Arginine (L-Arginine is prepared in 18.2 MOhm water), 10 uL of glacial acetic acid (1% v/v), 10 uL of 88% Formic Acid (1% v/v), 830 uL of 18.2 MOhm water. The PTFE sealed cap was placed on the 3 ml vial tightly. The solution and vial were placed into a conventional microwave and microwaved for 30 seconds at 50% power. After microwaving, the solution was placed into a centrifuge for 5 minutes to clarify. The supernatant was removed from any remaining pellet for spinning fibers or producing other materials such as films, gels or foams.

Fiber testing results (10 samples) 1.5× post spin stretch in an 80:20 isopropanol:water bath.

| | Diameter | Energy to Break | Max Stress | Max Strain |
|---|---|---|---|---|
| | (μm) | (MJ/m³) | (MPa) | (mm/mm) |
| Average | 43.25 | 2.25 | 31.68 | 0.08 |
| St. Dev. | 16.23 | 1.25 | 7.83 | 0.04 |

Fiber testing results (10 samples) 2.0× post spin stretch in an 80:20 isopropanol:water bath.

| | Diameter | Energy to Break | Max Stress | Max Strain |
|---|---|---|---|---|
| | (μm) | (MJ/m³) | (MPa) | (mm/mm) |
| Average | 30.02 | 5.02 | 61.68 | 0.09 |
| St. Dev. | 2.71 | 4.79 | 15.99 | 0.07 |

Fiber testing results (10 samples) 2.5× post spin stretch in an 80:20 isopropanol:water bath.

| | Diameter | Energy to Break | Max Stress | Max Strain |
|---|---|---|---|---|
| | (μm) | (MJ/m³) | (MPa) | (mm/mm) |
| Average | 28.44 | 20.93 | 73.15 | 0.30 |
| St. Dev. | 3.40 | 18.50 | 30.78 | 0.16 |

Fiber testing results (9 samples) 3.0× post spin stretch in an 80:20 isopropanol:water bath.

| | Diameter | Energy to Break | Max Stress | Max Strain |
|---|---|---|---|---|
| | (μm) | (MJ/m³) | (MPa) | (mm/mm) |
| Average | 27.57 | 3.85 | 33.38 | 0.14 |
| St. Dev. | 3.88 | 3.49 | 21.80 | 0.08 |

### Example Set 6

125 mg's of M4 (Nephila clavipes MaSP1 analogue) was measured out using a fine balance into a 3 ml Wheaton glass vial with PTFE seal inside a plastic cap. Included in the dope solution was 50 uL of 1M L-Arginine (L-Arginine is prepared in 18.2 MOhm water), 10 uL of glacial acetic acid (1% v/v), 30 uL of 88% Formic Acid (3% v/v), and 810 uL of 18.2 MOhm water. The PTFE sealed cap was placed on the 3 ml vial tightly. The solution and vial were placed into a conventional microwave and microwaved for 30 seconds. The solution and vial were allowed to cool and then, the solution was sonicated using a microtip on a Misonix sonicator for 1 minute at a power setting of 1.5. The PTFE sealed cap was placed on the 3 ml vial tightly. The solution and vial were placed into a conventional microwave and microwaved for 30 seconds at 50% power. After microwaving, the solution was placed into a centrifuge for 5 minutes to clarify. The supernatant was removed from any remaining pellet for spinning fibers or producing other materials such as films, gels or foams.

Fiber testing results (8 samples) 1.5× post spin stretch in an 80:20 isopropanol:water bath.

| | Diameter | Energy to Break | Max Stress | Max Strain |
|---|---|---|---|---|
| | (μm) | (MJ/m³) | (MPa) | (mm/mm) |
| Average | 37.05 | 0.44 | 58.36 | 0.01 |
| St. Dev. | 3.32 | 0.14 | 13.03 | 0.002 |

Fiber testing results (9 samples) 2.0× post spin stretch in an 80:20 isopropanol:water bath.

| | Diameter | Energy to Break | Max Stress | Max Strain |
|---|---|---|---|---|
| | (μm) | (MJ/m³) | (MPa) | (mm/mm) |
| Average | 49.03 | 0.51 | 33.61 | 0.02 |
| St. Dev. | 2.45 | 0.16 | 3.24 | 0.006 |

Fiber testing results (10 samples) 2.5× post spin stretch in an 80:20 isopropanol:water bath.

| | Diameter | Energy to Break | Max Stress | Max Strain |
|---|---|---|---|---|
| | (μm) | (MJ/m³) | (MPa) | (mm/mm) |
| Average | 39.26 | 2.43 | 65.19 | 0.05 |
| St. Dev. | 10.08 | 1.96 | 35.24 | 0.04 |

Fiber testing results (10 samples) 3.0× post spin stretch in an 80:20 isopropanol:water bath.

| | Diameter | Energy to Break | Max Stress | Max Strain |
|---|---|---|---|---|
| | (μm) | (MJ/m³) | (MPa) | (mm/mm) |
| Average | 26.42 | 1.12 | 98.28 | 0.02 |
| St. Dev. | 2.27 | 0.19 | 13.89 | 0.002 |

### Example Set 7

50 mg's (5% w/v) of M4 (Nephila clavipes MaSP1 analogue) was measured out using a fine balance into a 3 ml Wheaton glass vial with PTFE seal inside a plastic cap. Included in the dope solution was 50 uL of 1M L-Arginine (L-Arginine is prepared in 18.2 MOhm water), 10 uL of glacial acetic acid (1% v/v), and 940 uL of 18.2 MOhm water. The PTFE sealed cap was placed on the 3 ml vial tightly. The solution and vial were placed into a conventional microwave and microwaved for 30 seconds at 50% power. After microwaving and cooling for 5 minutes, the solution was sonicated for 1 minute at 3.0 watts. After microwaving, the solution was placed into a centrifuge for 2 minutes to clarify. The supernatant was removed from any remaining pellet for spinning fibers or producing other materials such as films, gels or foams.

Fiber testing results 1.5× post spin stretch in an 80:20 isopropanol:water bath.

| | Diameter | Energy to Break | Max Stress | Max Strain |
|---|---|---|---|---|
| | (μm) | (MJ/m³) | (MPa) | (mm/mm) |
| Average | 26.35 | 0.74 | 59.87 | 0.02 |
| St. Dev. | 0.35 | 0.39 | 8.30 | 0.007 |

Fiber testing results 3.0× post spin stretch in an 80:20 isopropanol:water bath.

| | Diameter | Energy to Break | Max Stress | Max Strain |
|---|---|---|---|---|
| | (μm) | (MJ/m³) | (MPa) | (mm/mm) |
| Average | 17.30 | 16.31 | 112.10 | 0.16 |
| St. Dev. | 1.15 | 12.92 | 16.81 | 0.12 |

### Example Set 8

80 mg's (8% w/v) of M4 (Nephila clavipes MaSP1 analogue) in addition to 20 mg's (2% w/v) of M5 (Nephila clavipes MaSP2 analogue) was measured out using a fine balance into a 3 ml Wheaton glass vial with PTFE seal inside a plastic cap. Included in the dope solution was 50 uL of 1M L-Arginine (L-Arginine is prepared in 18.2 MOhm water), 50 uL of glacial acetic acid (5% v/v), and 940 uL of 18.2 MOhm water. The PTFE sealed cap was placed on the 3 ml vial tightly. The solution and vial were placed into a conventional microwave and microwaved for 35 seconds at 50% power. After microwaving, the solution was placed into a centrifuge for 3 minutes to clarify. The supernatant was removed from any remaining pellet for spinning fibers or producing other materials such as films, gels or foams

Fiber testing results 2.0× post spin stretch in an 80:20 isopropanol:water bath.

| | Diameter | Energy to Break | Max Stress | Max Strain |
|---|---|---|---|---|
| | (μm) | (MJ/m³) | (MPa) | (mm/mm) |
| Average | 31.62 | 1.70 | 68.16 | 0.04 |
| St. Dev. | 5.59 | 0.42 | 14.59 | 0.003 |

Fiber testing results 2.5× post spin stretch in an 80:20 isopropanol:water bath.

| | Diameter | Energy to Break | Max Stress | Max Strain |
|---|---|---|---|---|
| | (μm) | (MJ/m³) | (MPa) | (mm/mm) |
| Average | 29.05 | 10.63 | 80.98 | 0.16 |
| St. Dev. | 1.07 | 3.84 | 10.78 | 0.04 |

Fiber testing results 3.0× post spin stretch in an 80:20 isopropanol:water bath.

| | Diameter | Energy to Break | Max Stress | Max Strain |
|---|---|---|---|---|
| | (μm) | (MJ/m³) | (MPa) | (mm/mm) |
| Average | 25.48 | 23.31 | 84.91 | 0.31 |
| St. Dev. | 1.85 | 17.46 | 6.96 | 0.23 |

Fiber testing results 3.5× post spin stretch in an 80:20 isopropanol:water bath.

| | Diameter | Energy to Break | Max Stress | Max Strain |
|---|---|---|---|---|
| | (μm) | (MJ/m³) | (MPa) | (mm/mm) |
| Average | 27.60 | 14.92 | 79.61 | 0.21 |
| St. Dev. | 3.88 | 10.83 | 37.40 | 0.13 |

### Example Set 9

62.5 mg's of M4 (Nephila clavipes MaSP1 analogue) and 62.5 mg's of M5 (Nephila clavipes MaSP2 analogue) was measured out using a fine balance into a 3 ml Wheaton glass vial with PTFE seal inside a plastic cap. Included in the dope solution was 18.2 MOhm water. The PTFE sealed cap was placed on the 3 ml vial tightly. The solution and vial were placed into a conventional microwave and microwaved for 5 seconds repetitively with mixing between bursts of microwave 5 total times. The solution and vial were allowed to cool and then, the solution was sonicated using a microtip on a Misonix sonicator for 1 minute at a power setting of 1.5. The PTFE sealed cap was placed on the 3 ml vial tightly. After microwaving, the solution was placed into a centrifuge for 5 minutes to clarify. The supernatant was removed from any remaining pellet for spinning fibers or producing other materials such as films, gels or foams.

Fiber testing results for a dual stretch; 2× then 1.5× post spin stretch in an 80:20 isopropanol:water and then 20:80 isopropanol:water bath.

| | Diameter | Energy to Break | Max Stress | Max Strain |
|---|---|---|---|---|
| | (μm) | (MJ/m³) | (MPa) | (mm/mm) |
| Average | 54.94 | 16.15 | 43.14 | 0.44 |
| St. Dev. | 2.55 | 7.88 | 4.87 | |

Fiber testing results for a dual stretch; 1.5× then 2.0× post spin stretch in an 80:20 isopropanol:water and then 20:80 isopropanol:water bath.

| | Diameter | Energy to Break | Max Stress | Max Strain |
|---|---|---|---|---|
| | (μm) | (MJ/m³) | (MPa) | (mm/mm) |
| Average | 45.41 | 8.12 | 54.24 | 0.17 |
| St. Dev. | 7.43 | 9.28 | 16.69 | 0.18 |

112.5 mg's of M4 (Nephila clavipes MaSP1 analogue) and 12.5 mg's of M5 (Nephila clavipes MaSP2 analogue) was measured out using a fine balance into a 3 ml Wheaton glass vial with PTFE seal inside a plastic cap. Included in the dope solution was 18.2 MOhm water, 0.1% v/v propionic acid, and 10 mM imidazole. The PTFE sealed cap was placed on the 3 ml vial tightly. The solution and vial were placed into a conventional microwave and microwaved for 5 seconds repetitively with mixing between bursts of microwave 5 total times. The solution and vial were allowed to cool and then, the solution was sonicated using a microtip on a Misonix sonicator for 1 minute at a power setting of 1.5. The PTFE sealed cap was placed on the 3 ml vial tightly. After microwaving, the solution was placed into a centrifuge for 5 minutes to clarify. The supernatant was removed from any remaining pellet for spinning fibers or producing other materials such as films, gels or foams.

Fiber testing results for a dual stretch; 2.0× then 2.5× post spin stretch in an 80:20 isopropanol:water and then 20:80 isopropanol:water bath.

| | Diameter | Energy to Break | Max Stress | Max Strain |
|---|---|---|---|---|
| | (μm) | (MJ/m³) | (MPa) | (mm/mm) |
| Average | 37.29 | 33.77 | 134.50 | 0.28 |
| St. Dev. | 2.64 | 33.55 | 38.78 | 0.26 |

Fiber testing results for a dual stretch; 2.5× then 2.0× post spin stretch in an 80:20 isopropanol:water and then 20:80 isopropanol:water bath.

| | Diameter | Energy to Break | Max Stress | Max Strain |
|---|---|---|---|---|
| | (μm) | (MJ/m³) | (MPa) | (mm/mm) |
| Average | 36.98 | 28.52 | 192.22 | 0.18 |
| St. Dev. | 3.84 | 11.97 | 51.74 | 0.04 |

Adhesives

50 mg's of M4 (Nephila clavipes MaSP1 analogue) was measured out using a fine balance into a 3 ml Wheaton glass vial with PTFE seal inside a plastic cap. Included in the dope solution was 1000 μL of 18.2 MOhm water. The PTFE sealed cap was placed on the 3 ml vial tightly. The solution and vial were placed into a conventional microwave and microwaved for 55 seconds. 80 μl of solubilized M4 was removed from the vial and pipette onto an acrylic plastic plate and assembled with a second plate as in FIG. 2. Half of the tested assemblies were scored plastic while the second half was unscored, smooth plastic. Gluing boards were heated in an oven to dry for 24 hours at 30° C. Samples were then tested with a mechanical testing frame (MTS) as shown in FIG. 2 with Max Stress and Max Strain observations as reported in Table 2.

TABLE 2

| | Max Stress (Mpa) | Max Strain (mm/mm) |
|---|---|---|
| Scored 0 | 0.240553633 | 0.061867451 |
| Scored 1 | 0.284113645 | 0.050748235 |
| Scored 2 | 0.290257593 | 0.032738039 |
| Scored 3 | 0.207217517 | 0.053287059 |
| Scored 4 | 0.275858824 | 0.038258039 |
| Scored 5 | 0.23692426 | 0.035489412 |
| Average | 0.255820912 | 0.045398039 |
| Unscored 1 | 0.045467462 | 0.018403922 |
| Unscored 2 | 0.087084871 | 0.039007059 |
| Unscored 3 | 0.108057714 | 0.051941961 |
| Unscored 4 | 0.115011765 | 0.027708235 |
| Unscored 6 | 0.152559701 | 0.064506667 |
| Average | 0.101636302 | 0.040313569 |

25 mg's of M4 (Nephila clavipes MaSP1 analogue) and 25 mg's of M5 (Nephila clavipes MaSP2 analogue) was measured out using a fine balance into a 3 ml Wheaton glass vial with PTFE seal inside a plastic cap. Included in the dope solution was 1000 μL of 18.2 MOhm water. The PTFE sealed cap was placed on the 3 ml vial tightly. The solution and vial were placed into a conventional microwave and microwaved for 55 seconds. 80 μl of solubilized M4 and M5 was removed to a plastic plate and assembled with a second plate as in FIG. 1. Half of the tested assemblies were scored plastic while the second half was unscored, smooth plastic. Gluing boards were heated in an oven to dry for 24 hours at 30° C. Samples were then tested with a mechanical testing frame (MTS) as represented in FIG. 2 with Max Stress and Max Strain observations as reported in Table 3.

TABLE 3

| | Max Stress (Mpa) | Max Strain (mm/mm) |
|---|---|---|
| Scored 0 | 0.405474817 | 0.059890196 |
| Scored 1 | 0.355278111 | 0.051212549 |
| Scored 2 | 0.509896194 | 0.055307451 |
| Scored 3 | 0.35507744 | 0.064501176 |
| Scored 4 | 0.300653595 | 0.038214902 |
| Scored 5 | 0.406243752 | 0.052461176 |
| Average | 0.388770652 | 0.053597908 |
| Unscored 1 | 0.267291844 | 0.049407843 |
| Unscored 2 | 0.336299834 | 0.05244549 |
| Unscored 3 | 0.287887532 | 0.04240549 |
| Unscored 4 | 0.341145098 | 0.051715294 |
| Unscored 6 | 0.336461433 | 0.038015686 |
| Average | 0.313817148 | 0.046797961 |

40 mg's of M4 (Nephila clavipes MaSP1 analogue) and 10 mg's of M5 (Nephila clavipes MaSP2 analogue) was measured out using a fine balance into a 3 ml Wheaton glass vial with PTFE seal inside a plastic cap.

Included in the dope solution was 1000 μL of 18.2 MOhm water. The PTFE sealed cap was placed on the 3 ml vial tightly. The solution and vial were placed into a conventional microwave and microwaved for 55 seconds. 80 μl of solubilized M4 and M5 was removed to a plastic plate and assembled with a second plate as in FIG. 1. Half of the tested assemblies were scored plastic while the second half was unscored, smooth plastic. Gluing boards were heated in an oven to dry for 24 hours at 30° C. Samples were then tested with a mechanical testing frame (MTS) as represented in FIG. 2 with Max Stress and Max Strain observations as reported in Table 4.

TABLE 4

| | Max Stress (Mpa) | Max Strain (mm/mm) |
|---|---|---|
| Scored 0 | 0.297577855 | 0.043788235 |
| Scored 1 | 0318335334 | 0.048578824 |
| Scored 2 | 0.480984237 | 0.074135686 |
| Scored 3 | 0.299748226 | 0.053203922 |
| Scored 4 | 0.360972549 | 0.049265882 |
| Scored 5 | 0.372502884 | 0.041647843 |
| Average | 0.355020181 | 0.051770065 |
| Unscored 1 | 0.123302074 | 0.019305882 |
| Unscored 2 | 0.185370089 | 0.037901961 |
| Unscored 3 | 0.107880133 | 0.025557647 |
| Unscored 4 | 0.184345098 | 0.031705882 |
| Unscored 6 | 0.167574578 | 0.031900392 |
| Average | 0.153694395 | 0.029274353 |

32.5 mg's of M4 (Nephila clavipes MaSP1 analogue) and 17.5 mg's of M5 (Nephila clavipes MaSP2 analogue) was measured out using a fine balance into a 3 ml Wheaton glass vial with PTFE seal inside a plastic cap. Included in the dope solution was 1000 μL of 18.2 MOhm water. The PTFE sealed cap was placed on the 3 ml vial tightly. The solution and vial were placed into a conventional microwave and microwaved for 90 seconds. 80 μl of solubilized M4 and M5 was removed to a plastic plate and assembled with a second plate as in FIG. 1. Half of the tested assemblies were scored plastic while the second half was unscored, smooth plastic. Gluing boards were heated in an oven to dry for 24 hours at 30° C. Samples were then tested with a mechanical testing frame (MTS) as represented in FIG. 2 with Max Stress and Max Strain observations as reported in Table 5.

TABLE 5

| | Max Stress (Mpa) | Max Strain (mm/mm) |
|---|---|---|
| Scored 0 | 0.355832372 | 0.048123137 |
| Scored 1 | 0.248919646 | 0.039603137 |
| Scored 2 | 0.516693579 | 0.078197647 |
| Scored 3 | 0.298771649 | 0.043566275 |
| Scored 4 | 0.230065359 | 0.033040784 |
| Scored 5 | 0.279468109 | 0.031095686 |
| Average | 0.321625119 | 0.045604444 |
| Unscored 1 | 0.114691674 | 0.024512157 |
| Unscored 2 | 0.21934737 | 0.040428235 |
| Unscored 3 | 0.212504624 | 0.038014118 |
| Unscored 6 | 0.232669566 | 0.037479216 |
| Average | 0.194803309 | 0.035108431 |

32.5 mg's of M4 (Nephila clavipes MaSP1 analogue) and 17.5 mg's of M5 (Nephila clavipes MaSP2 analogue) was measured out using a fine balance into a 3 ml Wheaton glass vial with PTFE seal inside a plastic cap. Included in the dope solution was 1000 μL, of 18.2 MOhm water. The PTFE sealed cap was placed on the 3 ml vial tightly. The solution and vial were placed into a conventional microwave and microwaved for 90 seconds. 80 μl of solubilized M4 and M5 was removed to a plastic plate and assembled with a second plate as in FIG. 1. Half of the tested assemblies were scored plastic while the second half was unscored, smooth plastic. Gluing boards were heated in an oven to dry for 24 hours at 30° C. Samples were then tested with a mechanical testing frame (MTS) as represented in FIG. 2 with Max Stress and Max Strain observations as reported in Table 6.

TABLE 6

| | Max Stress (Mpa) | Max Strain (mm/mm) |
|---|---|---|
| Scored 0 | 0.339623222 | 0.043734118 |
| Scored 1 | 0.323233293 | 0.040177255 |
| Scored 2 | 0.533025759 | 0.05397098 |
| Scored 3 | 0.307164111 | 0.03688 |
| Scored 4 | 0.29905421 | 0.041872941 |
| Scored 5 | 0.348283375 | 0.035167843 |
| Average | 0.358397328 | 0.04196719 |
| Unscored 1 | 0.083830634 | 0.022381961 |
| Unscored 2 | 0.141176471 | 0.034233725 |
| Unscored 3 | 0.107584166 | 0.029144314 |
| Unscored 4 | 0.184094118 | 0.047931765 |
| Unscored 6 | 0.149996185 | 0.021408627 |
| Average | 0.133336315 | 0.031020078 |

40 mg's of M4 (Nephila clavipes MaSP1 analogue) and 10 mg's of M5 (Nephila clavipes MaSP2 analogue) was measured out using a fine balance into a 3 ml Wheaton glass vial with PTFE seal inside a plastic cap. Included in the dope solution was 900 μL, of 18.2 MOhm water, 100 μl 1M imidazole [10 mM imidazole], and 1 μl propionic acid (99%). The PTFE sealed cap was placed on the 3 ml vial tightly. The solution and vial were placed into a conventional microwave and microwaved for 57 seconds. 80 μl of solubilized M4 and M5 was removed to a plastic plate and assembled with a second plate as in FIG. 1. Half of the tested assemblies were scored plastic while the second half was unscored, smooth plastic. Gluing boards were heated in an oven to dry for 24 hours at 30° C. Samples were then tested with a mechanical testing frame (MTS) as represented in FIG. 2 with Max Stress and Max Strain observations as reported in Table 7.

TABLE 7

| | Max Stress (Mpa) | Max Strain (mm/mm) |
|---|---|---|
| Scored 0 | 0.191741638 | 0.028792941 |
| Scored 1 | 0.201488595 | 0.034137255 |
| Scored 2 | 0.300499808 | 0.057567843 |
| Scored 3 | 0.198794537 | 0.039624314 |
| Scored 5 | 0.322301061 | 0.048321569 |
| Average | 0.242965128 | 0.041688784 |
| Unscored 1 | 0.097300369 | 0.026557647 |
| Unscored 2 | 0.064799942 | 0.024059608 |
| Unscored 3 | 0.074110248 | 0.02491451 |
| Unscored 4 | 0.038901961 | 0.006124706 |
| Unscored 6 | 0.171053635 | 0.032146667 |
| Average | 0.089233231 | 0.022760627 |

25 mg's of M4 (Nephila clavipes MaSP1 analogue) and 25 mg's of M5 (Nephila clavipes MaSP2 analogue) was measured out using a fine balance into a 3 ml Wheaton glass vial with PTFE seal inside a plastic cap. Included in the dope solution was 900 μL, of 18.2 MOhm water, 100 μl 1M imidazole [10 mM imidazole], and 1 μl propionic acid (99%). The PTFE sealed cap was placed on the 3 ml vial tightly. The suspension was sonicated for 1 minute at 3 watts using a microtip on a misonix 3000 sonicator. The solution and vial were placed into a conventional microwave and microwaved for 57 seconds. The solution was clarified by centrifugation at 18 kG for one minute. 80 μl of solubilized M4 and M5 was removed to a plastic plate and assembled with a second plate as in FIG. 1. Half of the tested assemblies were scored plastic while the second half was unscored, smooth plastic. Gluing boards were heated in an oven to dry for 24 hours at 30° C. Samples were then tested with a mechanical testing frame (MTS) as represented in FIG. 2 with Max Stress and Max Strain observations as reported in Table 8.

TABLE 8

| | Max Stress (Mpa) | Max Strain (mm/mm) |
|---|---|---|
| Scored 0 | 0.308066128 | 0.032904314 |
| Scored 1 | 0.310748299 | 0.038625882 |
| Scored 2 | 0.408981161 | 0.066913725 |
| Scored 4 | 0.291878897 | 0.048646275 |
| Scored 5 | 0.347314578 | 0.046457255 |
| Average | 0.333397813 | 0.04670949 |
| Unscored 1 | 0.082512119 | 0.022306667 |
| Unscored 2 | 0.120281169 | 0.029595294 |
| Unscored 3 | 0.173145098 | 0.034516078 |
| Unscored 4 | 0.163302052 | 0.040027451 |
| Unscored 6 | 0.184390619 | 0.036623529 |
| Average | 0.144726212 | 0.032613804 |

Silicon Adhesive

180 mg's of M4 (Nephila clavipes MaSP1 analogue) and 180 mg's of M5 (Nephila clavipes MaSP2 analogue) was measured out using a fine balance into a 8 ml Wheaton glass vial with PTFE seal inside a plastic cap. Included in the dope solution was 2640 μL of 18.2 MOhm water, 30 μl L-Dopa [stock concentration=10 mg/ml], 30 μl propionic acid (99%), 300 μl of imidazole [stock concentration=100 mM]. The PTFE sealed cap was placed on the 3 ml vial tightly. The suspension was sonicated for 1 minute at 3 watts using a microtip on a misonix 3000 sonicator. The solution and vial were placed into a conventional microwave and microwaved in 5 second burst until solubilized with mixing by swirling between bursts. The solution was clarified by centrifugation at 18 kG for one minute. Solubilized protein is then transferred to an air sprayer bowl (Master Airbrush® Brand Model VC16-B22). Air pressure is applied and a fine mist is produced. The mist is then coated onto each silicon surface to be adhered 3 times with a 3 minute dry period between coats.

A second dope is prepared as described in the first paragraph under the heading “Silicon Adhesive” as the bulk adhesive. Approximately 100 μl of that solution is then placed on top of one half of the coated silicon. The two pieces of silicon were then gently pressed together and placed into a drying oven preheated to 30° C. Adhesives were cured for 24 hours in the oven. Mechanical testing was performed on a MTS Synergy 100 by placing the ends of each piece of silicon in clamping grips and pulling on the ends until the bond failed.

| | Max stress | Max Strain | Energy to Break |
|---|---|---|---|
| Adhesive | (MPa) | (mm/mm) | (MJ/m³) |
| Super Glue | 0.0495 | 0.2099 | 0.0064 |
| Elmers | 0.0196 | 0.0612 | 0.0007 |
| Spider Silk | 0.0292 | 0.0819 | 0.0014 |

Foams

144 mg's of M4 (Nephila clavipes MaSP1 analogue) and 36 mg's of M5 (Nephila clavipes MaSP2 analogue) was measured out using a fine balance into a 8 ml Wheaton glass vial with PTFE seal inside a plastic cap.

Included in the dope solution was 2640 μL of 18.2 MOhm water, 30 μl L-Dopa [stock concentration=10 mg/ml], 30 μl propionic acid (99%), and 300 μl of imidazole [stock concentration=1M]. The PTFE sealed cap was placed on the 3 ml vial tightly. The suspension was sonicated for 1 minute at 3 watts using a microtip on a misonix 3000 sonicator. The solution and vial were placed into a conventional microwave and microwaved in 5 second burst until solubilized with mixing by swirling between bursts. The solution was clarified by centrifugation at 18 kG for one minute. The solubilized protein was then placed on a glass slide and aspirated with a glass pipette until air bubbles were dispersed throughout the solution and allowed to dry on the bench. After a several hours of drying, a spongy foam remained on the glass slide.

Hydrogels

60 mg's of M4 (Nephila clavipes MaSP1 analogue) and 60 mg's of M5 (Nephila clavipes MaSP2 analogue) was measured out using a fine balance into an 8 ml Wheaton glass vial with PTFE seal inside a plastic cap.

Included in the dope solution was 1960 μL of 18.2 MOhm water and 40 μl propionic acid (99%). The PTFE sealed cap was placed on the 3 ml vial tightly. The solution and vial were placed into a conventional microwave and microwaved in 5 second burst, 6 times with mixing by swirling between. The suspension was sonicated for 1 minute at 3 watts using a microtip on a misonix 3000 sonicator. After 1 minute of cooling, the lid was removed and the solution poured into a circular plastic mold. The mold is sealed on the bottom with sheet silicon to prevent the solution from leaking out of the mold. The mold can be of any dimension or shape. The solution remains in the mold until hydrogel formation and then removed by pushing the hydrogel from the mold.

60 mg's of M4 (Nephila clavipes MaSP1 analogue) and 60 mg's of M5 (Nephila clavipes MaSP2 analogue) was measured out using a fine balance into an 8 ml Wheaton glass vial with PTFE seal inside a plastic cap.

Included in the dope solution was 1960 μL of 18.2 MOhm water and 60 μl propionic acid (99%).

The PTFE sealed cap was placed on the 3 ml vial tightly. The solution and vial were placed into a conventional microwave and microwaved in 5 second burst, 6 times with mixing by swirling between. The suspension was sonicated for 1 minute at 3 watts using a microtip on a misonix 3000 sonicator. After 1 minute of cooling, the lid was removed and the solution poured into a circular plastic mold. The mold is sealed on the bottom with sheet silicon to prevent the solution from leaking out of the mold. The mold can be of any dimension or shape. The solution remains in the mold until hydrogel formation and then removed by pushing the hydrogel from the mold.

Hydrogels were treated with various alcohols after formation by submerging the hydrogel in their respective solution for 60 minutes, then a water rinse for 60 minutes and their mechanical properties studied. They are reported in Table 9.

TABLE 9

| Post- | Max Stress | Max Strain | Elastic Modulus |
|---|---|---|---|
| Treatment | (MPa) | (mm/mm) | (MPa) |
| H20 | 0.01727 | 0.222752 | 0.078356 |
| 50/50 MeOH | 0.027454 | 0.201667 | 0.175385 |
| 50/50 IPA | 0.033131 | 0.250283 | 0.165239 |
| 50/50 EtOH | 0.033621 | 0.175943 | 0.228684 |

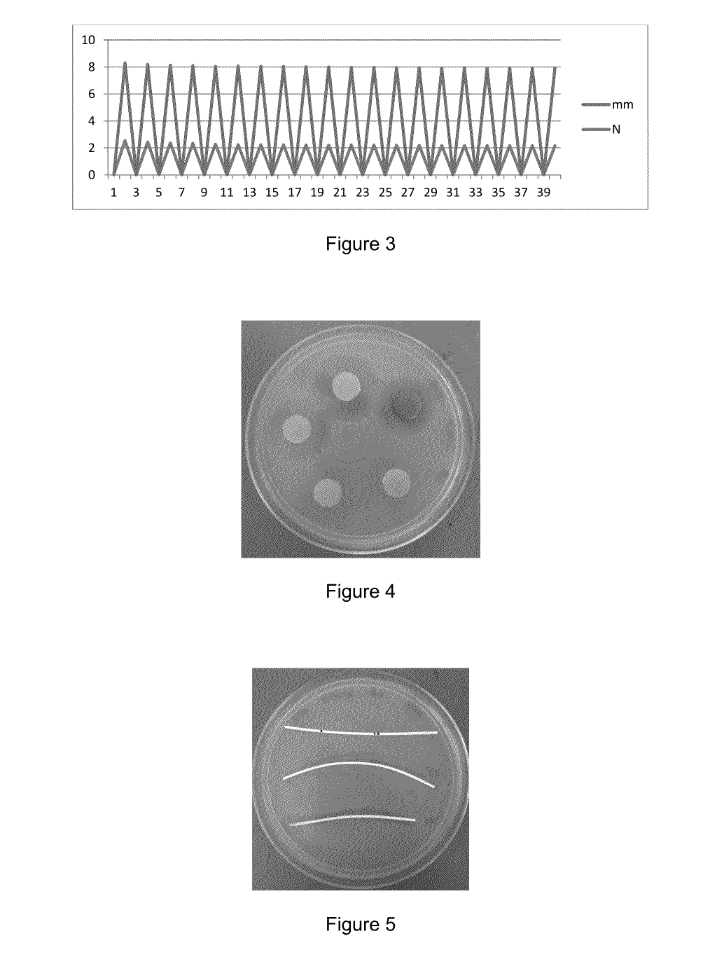

Hysteresis testing of hydrogels demonstrates that the gels produced are elastic and able to survive repeated loadings. Hydrogels were of the same formulation as reported in the first two paragraphs under heading “hydrogels” above. Results of testing hydrogels is shown in FIG. 3 as well as Table 10.

TABLE 10

| Specimen (No.) | Specimen Height (mm) | Peak Load for Entire Test (N) |
|---|---|---|
| 1 | 10.3 | 2.546 |
| 2 | 10.7 | 2.5 |
| 3 | 10.8 | 1.603 |
| 4 | 11 | 2.921 |

Lyogels

Lyogels are prepared from hydrogels prepared as reported. Once a hydrogel is formed, that gel is placed into a lypholization chamber and dried under vacuum until water is removed leaving only the protein behind. The lyogels can then be post treated to alter the mechanical properties of the lyogel.

60 mg's of M4 (Nephila clavipes MaSP1 analogue) and 60 mg's of M5 (Nephila clavipes MaSP2 analogue) was measured out using a fine balance into an 8 ml Wheaton glass vial with PTFE seal inside a plastic cap. Included in the dope solution was 1960 μL of 18.2 MOhm water and 40 μl propionic acid (99%). The PTFE sealed cap was placed on the 3 ml vial tightly. The solution and vial were placed into a conventional microwave and microwaved in 5 second burst, 6 times with mixing by swirling between. The suspension was sonicated for 1 minute at 3 watts using a microtip on a misonix 3000 sonicator. After 1 minute of cooling, the lid was removed and the solution poured into a circular plastic mold. The mold is sealed on the bottom with sheet silicon to prevent the solution from leaking out of the mold. The mold can be of any dimension or shape. The solution remains in the mold until hydrogel formation and then removed by pushing the hydrogel from the mold. Once the hydrogel was formed and removed from the mold, it was frozen and placed into a lypholization bell. Vacuum was applied for 12 hours. Once dried, the lyogels were removed from the lypholizer and treated with one of three alcohol solutions (50/50 water/isopropanol, 50/50 water/ethanol, 50/50 methanol) or water as a control and then mechanically tested by compressing them while measuring stress and strain as shown in Table 11.

TABLE 11

| Post | | Max Strain | Elastic Modulus |
|---|---|---|---|
| Treatment | Max Stress (MPa) | (mm/mm) | (MPa) |
| H20 | 0.93 | 0.76 | 1.00 |
| 50/50 IPA | 0.14 | 0.37 | 0.47 |
| 50/50 EtOH | 0.04 | 0.14 | 0.38 |
| 50/50 MeOH | 0.09 | 0.28 | 0.33 |

Coatings

90 mg's of M4 (Nephila clavipes MaSP1 analogue) and 90 mg's of M5 (Nephila clavipes MaSP2 analogue) was measured out using a fine balance into an 8 ml Wheaton glass vial with PTFE seal inside a plastic cap. Included in the dope solution was 2930 μl of 18.2 MOhm water, 10 mM imidazole, 30 μl propionic acid (99%), and 30 μl L-Dopa (30 ug L-Dopa). The suspension was sonicated for 1 minute at 3 watts using a microtip on a misonix 3000 sonicator. The PTFE sealed cap was placed on the 3 ml vial tightly. The solution and vial were placed into a conventional microwave and microwaved in 5 second burst, 6 times with mixing by swirling between. After 1 minute of cooling, the lid was removed and the solution removed to a either a dip bath or sprayer.

Dip coats were achieved by dipping samples repeatedly in the dope solution or dragging through a bath of spider silk protein. The samples are then dried completely on the bench. Dip coatings can be reapplied until the desired thickness is achieved.

Spray coatings were achieved by removing the soluble silk protein to a Master Airbrush Model G233 with a 0.2 mm needle tip and spraying the solution onto the substrate. Spray coatings can be reapplied until the desired thickness of coating is achieved.

Spray and dip coatings can be combined as well. It was observed that a light spray coating, after drying, then dip coating produced a visually impressive coating and it also appeared to adhere to the substrate to a greater degree.

Silicon wafers were coated with aqueous based recombinant spider silk proteins via the spraying method described in the first two paragraphs under the heading “Silicon Adhesives”. Antibiotics and other substances were included in the dope solutions to functionalize the coating. (Kanamycin at 10 μg/wafer). Coatings were submerged in Congo Red dye (β-sheet specific dye) to allow visualization of the coating without the aid of a microscope.

All solutions were prepared as dip coats described in the second paragraph following the heading “Coatings.” Resulting products are shown in FIG. 4.

Silicon urinary catheters (3 french) coated with spider silk protein (top), spider silk coating loaded with 50 mg/L kanamycin (middle) and spider silk coating loaded with 500 mg/L kanamycin (bottom) as shown in FIG. 5.

Stainless steel can also be coated using dope solutions prepared as described in the second paragraph following the heading “Coatings.” Both spray and dip coating can be used to coat surgical stainless steel.

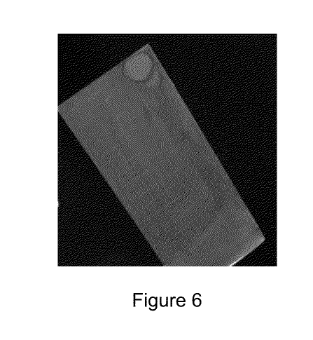

A stainless steel plate was dip coated with recombinant spider silk protein. Congo Red dye (stains β-sheets) was used to visualize the coating as shown in FIG. 6.

Capsules

Capsules can be generated from the described aqueous methods by solvating the recombinant spider silk proteins in water and then allowing them to precipitate or by driving their precipitation via salt precipitation. When combined with another substance, such as a vaccine, the spider silk proteins encapsulate the vaccine.

Alternative Solvation Method: Autoclave

An alternative method of solvation was also tested successfully. Rather than using a microwave to irradiate an aqueous dope, heat and pressure inside of a sealed vial was performed using an autoclave.

60 mg's of M4 (Nephila clavipes MaSP1 analogue) and 60 mg's of M5 (Nephila clavipes MaSP2 analogue) was measured out using a fine balance into an 8 ml Wheaton glass vial with PTFE seal inside a plastic cap.

Included in the dope solution was 1960 μL of 18.2 MOhm water and 40 μl propionic acid (99%). The PTFE sealed cap was placed on the 3 ml vial tightly. The solution and vial were placed into a conventional microwave and microwaved in 5 second burst, 6 times with mixing by swirling between. The suspension was sonicated for 1 minute at 3 watts using a microtip on a misonix 3000 sonicator. After 1 minute of cooling, the lid was removed and the solution poured into a circular plastic mold. The mold is sealed on the bottom with sheet silicon to prevent the solution from leaking out of the mold. The mold can be of any dimension or shape. The solution remains in the mold until hydrogel formation and then removed by pushing the hydrogel from the mold.

The vial was placed it an autoclave for 75 minutes at 123° C. and 20.1 PSI with the lid on, but not tightened. Immediately after removal from the autoclave and cooling, the solution turned to a hydrogel even though not all of the protein was solvated (visual inspection of white precipitate in vial). However, the method did work to solvate the protein as indicated by the formation of a hydrogel.

The autoclave experiment demonstrates that microwave irradiation unexpectantly provides conditions for aqueous solvation of rSSP. Without wishing to be bound to any particular theory, the source of temperature and pressure from microwave irradiation may be uniquely suited for solvation of the proteins. Microwave irradiation is convenient as it develops heat and temperature quickly within the vial while an autoclave took 75 minutes to only partially solubilize available protein. Other methods of generating heat and pressure are available that generate higher pressure and temperature without the use of a microwave that could be used to solubilize the proteins.

The methods and compositions described herein may also be applied to other traditionally insoluble proteins. Exemplary proteins that may be used in these methods include naturally occurring and synthetic proteins associated with protein misfolding diseases such as prions (CWD, BSE, vBSE, Creutzfeldt-Jakob), Alzheimers, and Parkinsons.

Additionally, synthetically produced G-protein couple receptors (GPCR) are difficult targets as they to suffer aqueous solubility issues. Approximately 40% of drugs produced today are targeted at GPCR's. The methods described herein may also be applied to such GPCR's.

In addition, numerous proteins when expressed in E. coli, are recovered as inclusion bodies. Inclusion bodies are aggregates of the expressed protein that are also insoluble in aqueous solutions. In order to solubilize these proteins, generally high concentrations of urea are used to denature the protein(s). Once denatured, the proteins then have to be renatured into their correct conformation for them to have biological activity. That is not an easy, cheap or quick means by which to synthetically produce proteins. The methods and compositions described herein may also address such insolubility issues with such proteins associated with inclusion bodies.

### Statements

1. A method of solubilizing one or more recombinant spider silk proteins in an aqueous solution, comprising:

mixing the one or more recombinant spider silk proteins with water to form a mixture in a sealed container;

heating the mixture to form a solution.

2. The method of claim 1, wherein the heating is performed with microwave irradiation.

3. The method of any one of claims 1-2, further comprising sonicating the mixture.

4. The method of any one of claims 1-3, further comprising sonicating the solution.

5. The method of any one of claims 1-4, further comprising centrifuging the solution.

6. The method of any one of claims 1-5, further comprising providing additives for reducing gel formation in the solution.

7. The method of claim 6, wherein the additives are selected from the group consisting of: an acid, a base, free amino acids, surfactants, and combinations thereof.

8. The method of claim 6, wherein the additives are selected from the group consisting of: propionic acid, formic acid, acetic acid, ammonium hydroxide, L-arginine, L-glutamic acid, β-mercaptoethanol, dithiothreitol, and combinations thereof.

9. The method of any one of claims 1-8, wherein the one or more recombinant spider silk proteins are selected from the group consisting of: M4, M5, MaSP1, a MaSP1 analogue, MaSP2, an MaSP2 analogue, and combinations thereof.

10. The method of any one of claims 1-9, wherein the ratio of the one or more recombinant spider silk proteins to water in the mixture is from 1:10 to 1:2.

11. The method of any one of claims 1-10, further comprising obtaining a recombinant spider silk protein fiber from the mixture.

12. The method of any one of claims 1-11, further comprising stretching the fiber.

13. A recombinant spider silk protein material prepared according to any one of claims 1-12, having the form of a hydrogel, lyogel, film, coating, foam, fiber, and combinations thereof.

14. An aqueous solution of recombinant spider silk proteins, comprising: one or more recombinant spider silk proteins and water, wherein the amount of the one or more recombinant spider silk proteins is greater than about 2% w/v.

15. The aqueous solution of claim 14, wherein the amount of the one or more recombinant spider silk proteins is less than about 50% w/v.

16. The aqueous solution of any one of claims 14 and 15, further comprising one or more additives for reducing gel formation.

17. The aqueous solution of claim 16, wherein the one or more additives are selected from the group consisting of: an acid, a base, free amino acids, surfactants, and combinations thereof.

18. The aqueous solution of claim 16, wherein the one or more additives are selected from the group consisting of: propionic acid, formic acid, acetic acid, ammonium hydroxide, L-arginine, L-glutamic acid, β-mercaptoethanol, dithiothreitol, and combinations thereof.

It will be appreciated that variations of the above-disclosed and other features and functions, or alternatives thereof, may be desirably combined into many other different systems or applications. Also, various presently unforeseen or unanticipated alternatives, modifications, variations or improvements therein may be subsequently made by those skilled in the art, and are also intended to be encompassed by the following claims.

What is claimed is:

1. A method of solubilizing one or more recombinant spider silk proteins in an aqueous solution, comprising:
mixing the one or more recombinant spider silk proteins with water to form a mixture in a sealed container;
heating the mixture to form a solution.

2. The method of claim 1, wherein the heating is performed with microwave irradiation.

3. The method of claim 1, further comprising sonicating the mixture.

4. The method of claim 1, further comprising sonicating the solution.

5. The method of claim 1, further comprising centrifuging the solution.

6. The method of claim 1, further comprising providing additives for reducing gel formation in the solution.

7. The method of claim 6, wherein the additives are selected from the group consisting of: an acid, a base, free amino acids, surfactants, and combinations thereof.

8. The method of claim 6, wherein the additives are selected from the group consisting of: propionic acid, formic acid, acetic acid, ammonium hydroxide, L-arginine, L-glutamic acid, β-mercaptoethanol, dithiothreitol, and combinations thereof.

9. The method of claim 1, wherein the one or more recombinant spider silk proteins are selected from the group consisting of: M4, M5, MaSP1, a MaSP1 analogue, MaSP2, an MaSP2 analogue, and combinations thereof.

10. The method of claim 1, wherein the ratio of the one or more recombinant spider silk proteins to water in the mixture is from 1:10 to 1:2.

11. The method of claim 1, further comprising obtaining a recombinant spider silk protein fiber from the mixture.

12. The method of claim 11, further comprising stretching the fiber.

13. A recombinant spider silk protein material prepared according to claim 12, having the form of a hydrogel, lyogel, film, coating, foam, fiber, and combinations thereof.

14. An aqueous solution of recombinant spider silk proteins, comprising: one or more recombinant spider silk proteins and water, wherein the amount of the one or more recombinant spider silk proteins is greater than about 2% w/v.

15. The aqueous solution of claim 14, wherein the amount of the one or more recombinant spider silk proteins is less than about 50% w/v.

16. The aqueous solution of claim 14, further comprising one or more additives for reducing gel formation.

17. The aqueous solution of claim 16, wherein the one or more additives are selected from the group consisting of: an acid, a base, free amino acids, surfactants, and combinations thereof.

18. The aqueous solution of claim 16, wherein the one or more additives are selected from the group consisting of: propionic acid, formic acid, acetic acid, ammonium hydroxide, L-arginine, L-glutamic acid, β-mercaptoethanol, dithiothreitol, and combinations thereof.

19. A hydrogel made from mixing the one or more recombinant spider silk proteins with water to form a mixture in a sealed container and heating the mixture to form a solution.

20. The hydrogel of claim 19, further comprising an antibiotic material.
